(19) United States
(12) Patent Application Publication
(10) Pub. No.: US 2025/0095822 A1
(43) Pub. Date: Mar. 20, 2025

(54) PARALLEL GENERATION OF PARETO OPTIMAL RADIOTHERAPY PLANS

(71) Applicant: Elekta Instrument AB, Stockholm (SE)

(72) Inventors: Jens Olof Sjolund, Uppsala (SE); Carl Axel Håkan Nordström, Stockholm (SE)

(21) Appl. No.: 18/832,811

(22) PCT Filed: Feb. 11, 2022
(86) PCT No.: PCT/EP2022/053438

§ 371 Date: Jul. 24, 2024

(51) Int. Cl. G16H 20/40; A61N 5/10

(52) U.S. Cl. CPC G16H 20/40; A61N 5/103; A61N 5/1048; A61N 2005/1074

(57) ABSTRACT

Systems and methods are disclosed for dynamic adaptation of radiotherapy treatments. Example operations for radiotherapy treatment planning include: obtaining a radiotherapy problem (e.g., a multicriteria optimization problem) for providing radiotherapy treatment; performing treatment planning optimization for delivery of the radiotherapy treatment; and generating treatment plan data based on at least one of a plurality of solutions, with such treatment plan data being used to generate a radiation therapy treatment plan for a particular human subject with a radiotherapy machine. In an example, the treatment plan optimization includes identifying parameterized linear programming equations from the radiotherapy problem; converting the parameterized linear programming equations for execution by parallel processing hardware; and solving a plurality of the converted parameterized linear programming equations in parallel on the parallel processing hardware, to produce the plurality of solutions (e.g., encompassing the pareto surface) to the radiotherapy problem that correspond to the parameters that define the radiotherapy problem.

### TECHNICAL FIELD

Embodiments of the present disclosure pertain generally to processing and optimization techniques used in connection with a radiation therapy planning and treatment system. In particular, the present disclosure pertains to methods for the use of specific computing hardware configurations to identify optimized plans for a radiation therapy session.

### BACKGROUND

Radiation therapy (or “radiotherapy”) can be used to treat cancers or other ailments in mammalian (e.g., human and animal) tissue. One such radiotherapy technique is provided using a Gamma Knife, by which a patient is irradiated by a large number of low-intensity gamma rays that converge with high intensity and high precision at a target (e.g., a tumor). Another such radiotherapy technique is provided using a linear accelerator (Linac), whereby a tumor is irradiated by high-energy particles (e.g., electrons, protons, ions, high-energy photons, and the like). The placement and dose of the radiation beam must be accurately controlled to ensure the tumor receives the prescribed radiation, and the placement of the beam should be such as to minimize damage to the surrounding healthy tissue, often called the organ(s) at risk (OARs).

In radiotherapy, treatment plans are usually generated by solving an optimization problem that balances various conflicting objectives, such as high dose to target, normal tissue sparing, and treatment complexity. It is therefore a multicriteria optimization (MCO) problem. Commonly, the different criteria are combined using a weighted sum, where each weight determines the relative importance of that criterion. For a convex optimization problem, optimization attempts to identify all Pareto optimal radiotherapy plans. In this context, a Pareto optimal plan refers to radiotherapy plans where no criterion can be improved without worsening another.

Finding acceptable weights to develop a Pareto optimal radiotherapy plan is often a manual and tedious process of trial-and-error, especially because evaluating a single choice of parameters requires solving a full optimization problem for the radiotherapy treatment. Solving a full optimization problem for a single plan may take from a few seconds up to an hour to calculate and evaluate the parameter combination, depending on the application.

### OVERVIEW

Various embodiments, methods, systems, and computer-readable mediums are provided for the generation of radiotherapy plans, by solving a radiotherapy problem adjustable via parameters using an optimization method. This optimization method can produce a plurality of solutions which are then used to identify a parameter choice that results in a satisfactory radiotherapy plan.

The radiotherapy problem may be expressed as a multicriteria optimization (MCO) problem, where the parameters correspond to clinical preferences. The plurality of solutions may then be used to identify a Pareto surface (also known as the Pareto frontier). A Pareto optimal plan, as used herein, refers to a plan which is optimized such that no criterion can be improved without worsening another. The set of all Pareto optimal plans constitutes the Pareto surface.

The following provides an expanded approach for radiotherapy treatment planning optimization using a specialized configuration of a linear programming (LP) solver to identify the Pareto surface. In an example, a LP solver is adapted to apply an alternating direction method of multipliers (ADMM) technique which enables execution of the LP solver on parallel processing hardware such as GPUs. As a result, LP equations can be solved in parallel, in a substantially faster manner than being solved sequentially.

In various examples, operations for such radiotherapy treatment planning include: obtaining a radiotherapy problem for providing radiotherapy treatment to a human subject, the radiotherapy problem being adjustable via parameters; performing treatment planning optimization for delivery of the radiotherapy treatment, the treatment planning optimization comprising: (i) identifying parameterized linear programming equations from the radiotherapy problem; (ii) converting the parameterized linear programming equations for execution by parallel processing hardware; and (iii) solving a plurality of the converted parameterized linear programming equations in parallel on the parallel processing hardware, to produce a plurality of solutions (e.g., pareto-optimal solutions) to the radiotherapy problem corresponding to the parameters defined by the radiotherapy problem; and generating treatment plan data based on at least one of the plurality of solutions, with such treatment plan data being used to generate a radiation therapy treatment plan for a particular patient for radiation therapy via particular radiation therapy machine.

In further examples, the radiotherapy problem is a multicriteria optimization problem, and the parameters defined by the radiotherapy problem correspond to clinical preferences. Additionally, performing the treatment plan optimization for delivery of the radiotherapy treatment may include receiving a request to solve the radiotherapy problem, and receiving providing a plurality of sets of the parameters defined by the radiotherapy problem. For instance, a parameter in the plurality of sets of the parameters may concern a particular anatomical area of the human subject to receive the radiotherapy treatment from the radiotherapy treatment machine, or more specifically, the plurality of sets of the parameters may include definitions of one or more organ at risk areas and one or more target areas.

As noted, converting the linear programming equations may comprise applying an alternating direction method of multipliers (ADMM) technique. Specifically, the alternating direction method of multipliers technique may comprise transforming the linear programming equations to matrix and projection operations (e.g., as described with reference to Equation 6, below). The parallel processing hardware may comprise a plurality of graphics processing units (GPUs), which can execute the matrix operations in parallel.

In further examples, a representation of a solution space (e.g., the Pareto surface comprising a set of Pareto optimal solutions) to the radiotherapy problem may be generated and output based on the plurality of generated solutions. Other graphical representations may include generating a display of a graphical user interface, that is configured to provide functionality to configure the treatment plan, and displaying, within the graphical user interface, information associated with a solution to the radiotherapy problem for a particular set of parameters. Such information associated with the solution that is displayed may be based on the representation of the solution space to the radiotherapy problem (e.g., the Pareto surface) or characteristics of individual solutions. Additionally, such graphical user interface may be used for obtaining user interaction to modify the particular set of parameters.

In further examples, the operations for such radiotherapy treatment planning optimization further include: selecting a solution to the radiotherapy problem based on evaluation of the plurality of solutions; wherein the treatment plan data is generated based on the selected solution to the radiotherapy problem. For instance, the selected solution to the radiotherapy problem may provide an approximate solution, allowing the operations for the radiotherapy treatment planning optimization to include: receiving an additional optimization of the selected solution; wherein the treatment plan data for radiotherapy treatment is generated based on the additional optimization to the selected solution.

The treatment plan data for the radiotherapy treatment may comprise a set of treatment delivery parameters corresponding to capabilities of the radiotherapy treatment machine. In an example, the radiotherapy treatment is provided with a Gamma knife, and the set of treatment delivery parameters comprises a set of isocenters used for delivery of the radiotherapy treatment. For instance, the set of treatment delivery parameters further comprises timing for delivery of the radiotherapy treatment and a collimator sequence for the delivery of the radiotherapy treatment with the Gamma knife. As another example, the radiotherapy treatment is provided with a Volumetric-modulated arc therapy (VMAT) or Intensity modulated radiation therapy (IMRT), e.g., using a Linac radiotherapy machine, and the set of treatment delivery parameters comprises one or more of: a set of arc control points for one or more arcs, fluence fields, gantry speed, and dose rate along the one or more arcs.

In further examples, the operations may be followed by operations that cause or effect the delivery of the radiotherapy treatment using a plurality of radiotherapy beams from the radiotherapy treatment machine, based on the treatment plan data for the radiotherapy treatment.

The above overview is intended to provide an overview of subject matter of the present patent application. It is not intended to provide an exclusive or exhaustive explanation of the inventive subject matter. The detailed description is included to provide further information about the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates a radiotherapy system, according to some examples.

FIG. 2A illustrates a radiotherapy system having output configured to provide a therapy beam, according to some examples.

FIG. 2B illustrates a system including a combined radiation therapy system and an imaging system, such as a cone beam computed tomography (CBCT) imaging system, according to some examples.

FIG. 3 illustrates a partially cut-away view of a system including a combined radiation therapy system and an imaging system, such as a nuclear magnetic resonance (MR) imaging (MRI) system, according to some examples.

FIG. 4 illustrates an example of a Leksell Gamma Knife radiotherapy device, according to some examples.

FIG. 5 illustrates a radiotherapy treatment planning workflow using parallel processing for radiology problem optimization, according to some examples.

FIG. 6 illustrates a workflow for applying an alternating direction method of multipliers to parallelize execution of linear or quadradic programming equations, according to some examples.

FIG. 7 illustrates a flowchart for a method of radiotherapy treatment planning, according to some examples.

FIG. 8 illustrates an exemplary block diagram of a machine on which one or more of the methods as discussed herein can be implemented.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized and that structural, logical and electrical changes may be made without departing from the scope of the present disclosure. The following detailed description is, therefore, not to be taken in a limiting sense, and the scope of the present disclosure is defined by the appended claims and their equivalents.

The following discusses various implementations of planning and optimization techniques usable in radiotherapy or radiosurgery applications. Such techniques may be used to generate a large number of solutions to a radiotherapy problem, which may correspond to different parameter choices, and generate treatment plan data based on at least one of the found solutions. Such solutions may be solved in parallel on parallel processing hardware, to efficiently and effectively produce a plurality of solutions for the radiotherapy problem.

In the examples discussed herein, treatment planning problems are solved through use of a LP solver which operates based on the alternating direction method of multipliers (ADMM). This LP solver is able to solve a large number of problems in parallel on parallel processing hardware such as graphical processing units (GPUs). The use of an ADMM and parallel processing execution approaches can (approximately) solve all the LPs substantially faster than solving the LPs sequentially.

The consideration of a radiotherapy plan optimization problem as a LP problem, and the present techniques for transforming the LP problem into a parallel processing space, offers significant technical and clinical benefits. The technical benefits include reduced computing processing times to generate radiotherapy treatment plans, enhanced computation solutions for radiotherapy treatment plan optimization problems, and accompanying improvements in processing, memory, and network resources used to generate radiotherapy treatments.

As is explained in the sections below, the following techniques may be used to generate an entire Pareto surface of Pareto-optimal treatment plans, which are then selected and further optimized or validated for use with radiotherapy. Because the aim is to navigate the Pareto surface to identify a starting point for more detailed radiotherapy planning, the optimization problems do not need to be solved with high accuracy. Accordingly, in a radiotherapy planning setting (e.g., planning for a Gamma Knife treatment), a sufficiently accurate approximation of the Pareto optimal plans may be generated much faster than conventional approaches. This may enable use of a workflow where planning and treatment are performed on the same day.

Prior approaches for pre-calculating a representative set of treatment plans with conventional processing techniques often resulted in significant computation time and delays. The number of representative plans needed to span the Pareto surface for a Gamma Knife treatment is typically on the order of hundreds or thousands of plans, which means that a very long computation time (typically an over-night run) is required to generate a full representation of possible solutions. Some prior approaches also attempted to use AI-based methods for producing estimated solutions that solve the optimization problem. However, such AI-based methods require large amounts of training data to cover all edge cases and often require integration with a larger AI set of tools to be deployed in a product, even though the Pareto prediction for a new case is approximative.

Some dose optimization software has been designed to solve an optimization problem using a simplex solver for LP problems. A simplex solver is mathematical modeling technique in which a linear function is maximized or minimized when subjected to linear constraints. However, when using a simplex solver method, computations often cannot be batched together when solving several, closely related LP equations. This prevents the use of computation hardware designed for parallel execution (such as graphical processing units (GPUs)).

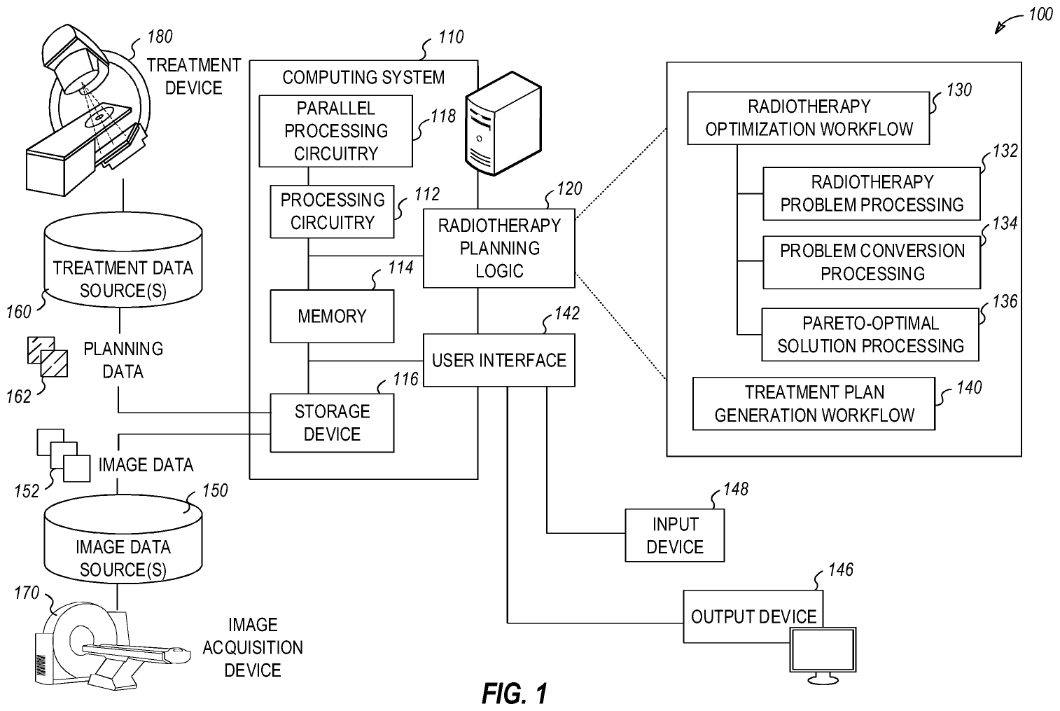

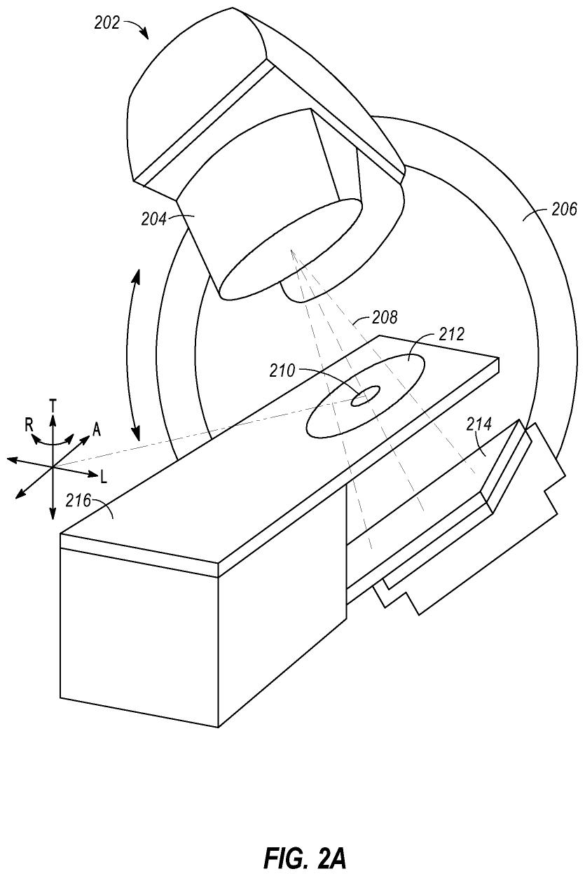

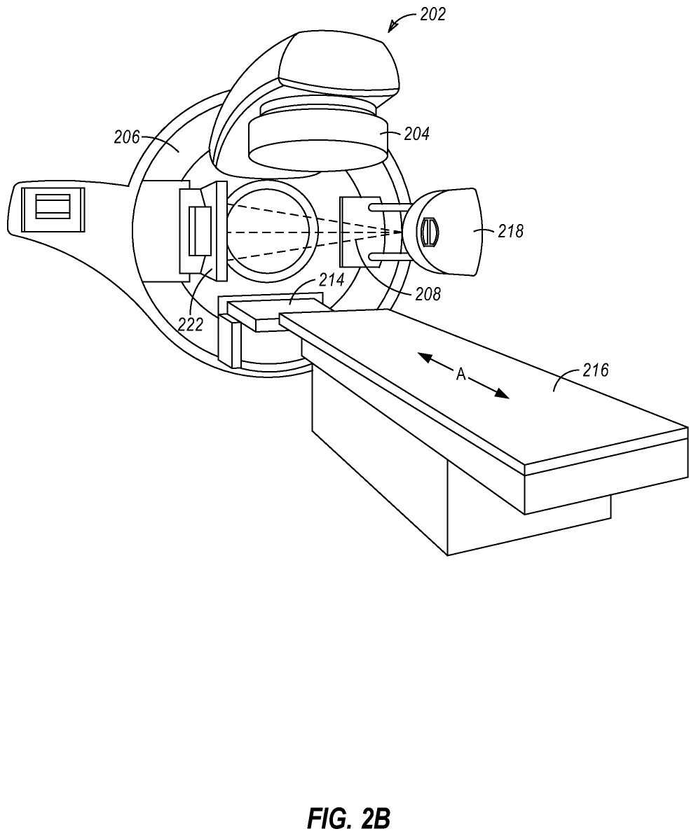

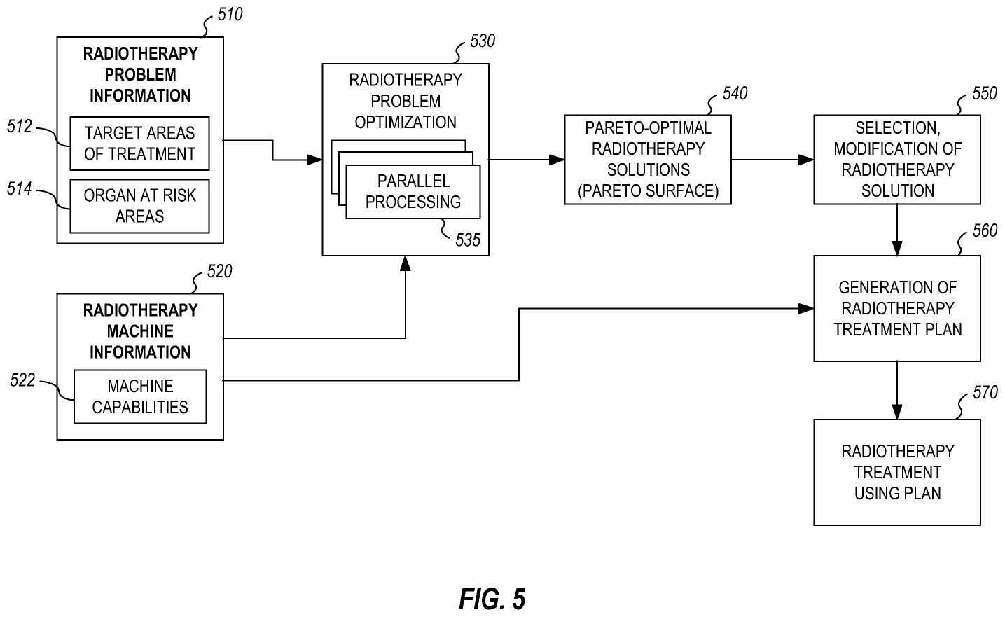

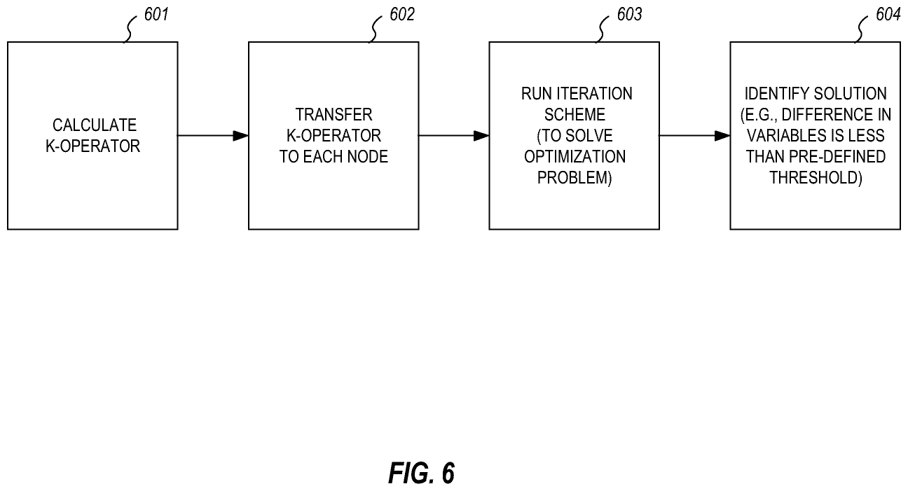

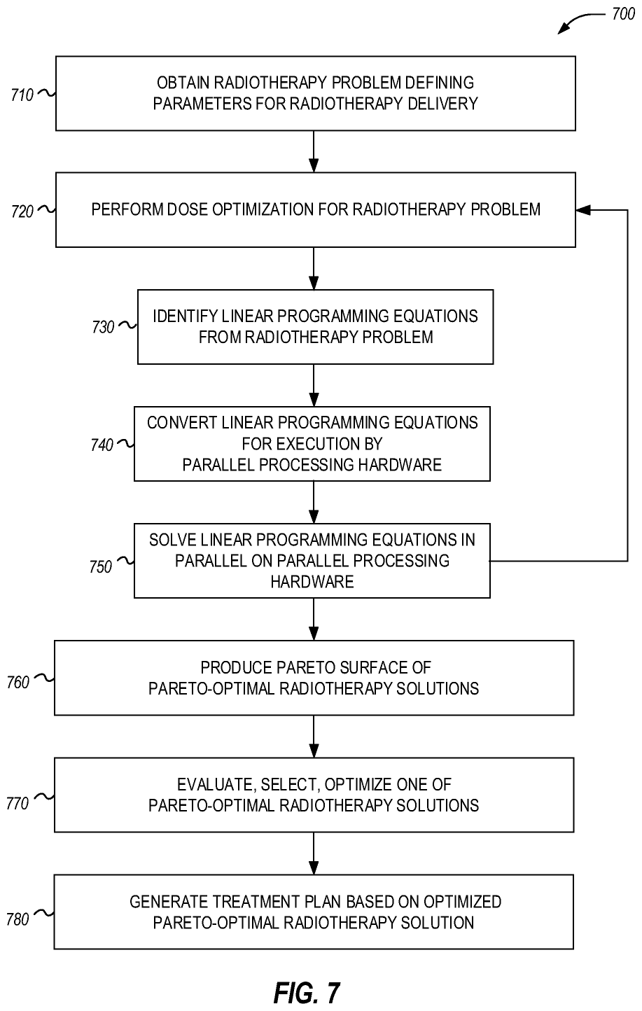

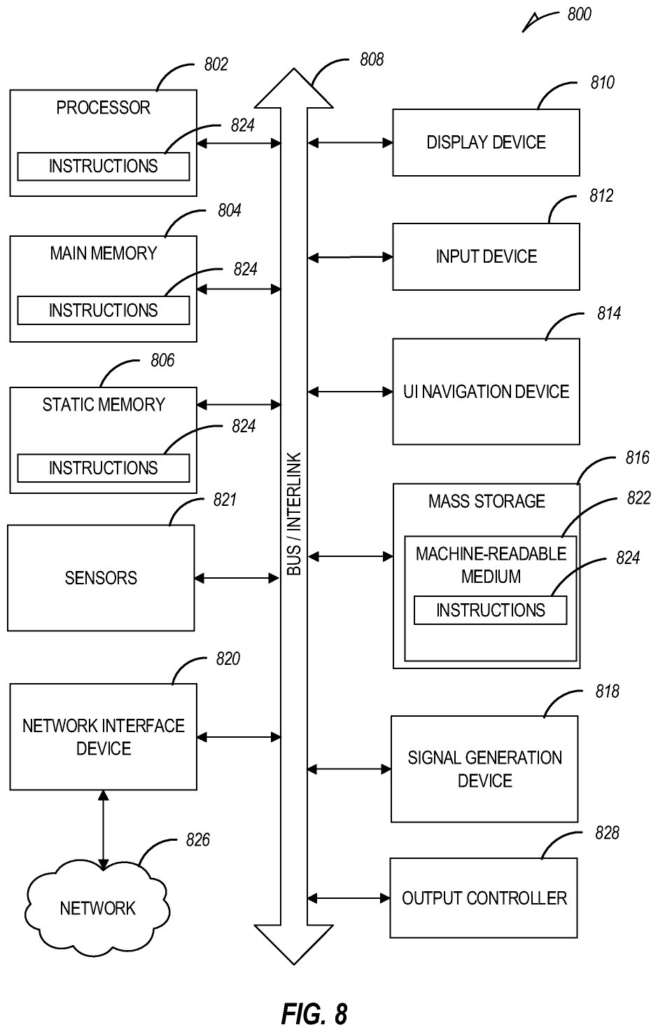

These and other limitations are addressed in the following configurations. The following paragraphs provide an overview of example radiotherapy system implementations and treatment use cases (with reference to FIGS. 2A, 2B, 3 and 4), including with the use of computing systems and hardware implementations (with reference to FIGS. 1 and 8). The following then continues with a discussion of example treatment planning and algorithm processing workflows (with reference to FIGS. 5 and 6). Finally, a discussion of radiotherapy treatment planning (with reference to FIG. 7) is provided, which illustrates an end-to-end method of generating an optimized treatment plan.

FIG. 1 illustrates an exemplary radiotherapy system 100 adapted to perform radiotherapy plan processing operations using one or more of the approaches discussed herein. These radiotherapy plan processing operations are performed to enable the radiotherapy system 100 to provide radiation therapy to a patient based on specific aspects of captured medical imaging data and therapy dose calculations or radiotherapy machine configuration parameters. Specifically, the following processing operations may be implemented as part of the radiotherapy planning logic 120 for developing a radiotherapy treatment plan. It will be understood, however, that many variations and use cases of the following planning logic 120 and optimization operations may be provided, such as in response to data verification, visualization, and other medical evaluative and diagnostic operations.

The radiotherapy system 100 includes a radiotherapy processing computing system 110 which hosts radiotherapy planning logic 120. The radiotherapy processing computing system 110 may be connected to a network (not shown), and such network may be connected to the Internet. For instance, a network can connect the radiotherapy processing computing system 110 with one or more private and/or public medical information sources (e.g., a radiology information system (RIS), a medical record system (e.g., an electronic medical record (EMR)/electronic health record (EHR) system), an oncology information system (OIS)), one or more image data sources 150, an image acquisition device 170 (e.g., an imaging modality), a treatment device 180 (e.g., a radiation therapy device), and a treatment data source 160.

As an example, the radiotherapy processing computing system 110 can be configured to receive a treatment goal of a subject (e.g., from one or more MR images) and generate a radiotherapy treatment plan by executing instructions or data from the radiotherapy planning logic 120, as part of operations to generate treatment plans to be used by the treatment device 180 and/or for output on device 146. In an embodiment, the radiotherapy planning logic 120 solves an optimization problem to generate the radiotherapy treatment plan. The radiotherapy planning logic 120 solves the radiotherapy optimization problem by estimating optimization variables of the received optimization problem. Then, the optimization problem is solved using a optimization problem solver. Such optimization problem solvers include, e.g., a simplex method, an interior point method, a Newton method, a quasi-Newton method, a Gauss-Newton method, a Levenberg-Marquardt method, a linear least-squares method, a gradient descent method, a projected gradient method, a conjugate gradient method, an augmented Lagrangian method, a Nelder-Mead method, a branch and bound method, a cutting plane method, simulated annealing, and/or sequential quadratic programming, or as discussed below, the use of ADMM applied on parallel processing circuitry 118.

A generic radiotherapy treatment plan optimization problem can be defined as Equation 1:

$$\begin{matrix}{minimize}_{x ∈ X} f ( x ) \\ subject to x ∈ Ω\end{matrix} \qquad ( Equation 1 )$$

where ƒ: X-> [symbol] is the objective function, x∈ X is the decision variables (also referred to as optimization variables) and Ω ⊆ X is the set of feasible variables. In general, the function ƒ can be nonlinear and the set Ω non-convex. The optimization problems are typically solved using some form of iterative scheme. For example, in case ƒ is smooth and convex, and Ω is convex, then the projected gradient scheme could be used to solve equation (1) and reads as follows:

$${x}_{n + 1} = {proj}_{Ω} ( {x}_{n} - η ∇ f ( {x}_{n} ) ) \qquad ( Equation 2 )$$

where proj_{Ω}: X->X is the projection onto Ω, η∈ [symbol] is a stepsize and ∇ƒ: X->X the gradient. While these algorithms are typically provably convergent (e.g., given enough time (and correct parameter choices), the algorithm will converge to a minimizer), they are not always very fast and efficient. In fact, several algorithms may require hundreds if not thousands of iterations in order to achieve approximate convergence. Since each step may be computationally expensive, this may imply runtimes of minutes or even hours.

According to the disclosed techniques, solutions to such optimization problems are produced by applying solver methods to solve the optimization problems with use of parallel processing hardware. In some scenarios, the optimization problems are solved within a deviation threshold of a desired or expected solution. The disclosed techniques enhance the speed and efficiency of solving the optimization problem by the parallel execution of the solver methods. As will be understood, methods used to solve an optimization problem will often apply an accuracy exit criteria (deviation threshold). However, the use of ADMM as discussed herein, in addition to being easily parallelizable, will quickly produce a reasonable and accurate solution. As a result, the solution produced by ADMM is likely to provide information that can be used to make a decision whether the solution is of clinically interest or not.

The radiotherapy processing computing system 110 may include processing circuitry 112, memory 114, a storage device 116, parallel processing circuitry 118, and other hardware and software-operable features such as a user interface 142, a communication interface (not shown), and the like. The storage device 116 may store transitory or non-transitory computer-executable instructions, such as an operating system, radiation therapy treatment plans, training data, software programs (e.g., image processing software, image or anatomical visualization software, artificial intelligence (AI) or ML implementations and algorithms such as provided by deep learning models, ML models, and neural networks (NNs), etc.), and any other computer-executable instructions to be executed by the processing circuitry 112.

In an example, the processing circuitry 112 may include a processing device, such as one or more general-purpose processing devices such as a microprocessor, a central processing unit (CPU), a graphics processing unit (GPU), an accelerated processing unit (APU), or the like. More particularly, the processing circuitry 112 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 112 may also be implemented by one or more special-purpose processing devices such as an application-specific integrated circuit (ASIC), a field programmable gate array (FPGA), a digital signal processor (DSP), a System on a Chip (SoC), or the like.

As would be appreciated by those skilled in the art, in some examples, the processing circuitry 112 may be a special-purpose processor rather than a general-purpose processor. The processing circuitry 112 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processing circuitry 112 may also include graphical processing units such as a GPU from the GeForce®, Quadro®, Tesla® family manufactured by Nvidia™, GMA, Iris™ family manufactured by Intel™, or the Radeon™ family manufactured by AMD™. The processing circuitry 112 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one physical (circuitry-based) or software-based processor (for example, a multi-core design or a plurality of processors each having a multi-core design). The processing circuitry 112 can execute sequences of transitory or non-transitory computer program instructions, stored in memory 114, and accessed from the storage device 116, to perform various operations, processes, and methods that will be explained in greater detail below. It should be understood that any component in system 100 may be implemented separately and operate as an independent device and may be coupled to any other component in system 100 to perform the techniques described in this disclosure.

The memory 114 may comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that may be used to store information including images, training data, one or more ML model(s) or technique(s) parameters, data, or transitory or non-transitory computer executable instructions (e.g., stored in any format) capable of being accessed by the processing circuitry 112, or any other type of computer device. For instance, the computer program instructions can be accessed by the processing circuitry 112, read from the ROM, or any other suitable memory location, and loaded into the RAM for execution by the processing circuitry 112.

The storage device 116 may constitute a drive unit that includes a transitory or non-transitory machine-readable medium on which is stored one or more sets of transitory or non-transitory instructions and data structures (e.g., software) embodying or utilized by any one or more of the methodologies or functions described herein (including, in various examples, the radiotherapy planning logic 120 and the user interface 142). The instructions may also reside, completely or at least partially, within the memory 114 and/or within the processing circuitry 112 during execution thereof by the radiotherapy processing computing system 110, with the memory 114 and the processing circuitry 112 also constituting transitory or non-transitory machine-readable media. The instructions may also cause the parallel processing circuitry 118 to perform specific processing operations.

The memory 114 and the storage device 116 may constitute a non-transitory computer-readable medium. For example, the memory 114 and the storage device 116 may store or load transitory or non-transitory instructions for one or more software applications on the computer-readable medium. Software applications stored or loaded with the memory 114 and the storage device 116 may include, for example, an operating system for common computer systems as well as for software-controlled devices. The radiotherapy processing computing system 110 may also operate a variety of software programs comprising software code for implementing the radiotherapy planning logic 120 and the user interface 142. Further, the memory 114 and the storage device 116 may store or load an entire software application, part of a software application, or code or data that is associated with a software application, which is executable by the processing circuitry 112. In a further example, the memory 114 and the storage device 116 may store, load, and manipulate one or more radiation therapy treatment plans, imaging data, segmentation data, treatment visualizations, histograms or measurements, one or more AI model data (e.g., weights and parameters of one or more ML model(s)), training data, labels and mapping data, and the like. It is contemplated that software programs may be stored not only on the storage device 116 and the memory 114 but also on a removable computer medium, such as a hard drive, a computer disk, a CD-ROM, a DVD, a Blu-Ray DVD, USB flash drive, a SD card, a memory stick, or any other suitable medium; such software programs may also be communicated or received over a network.

The parallel processing circuitry 118 may include any of the processing circuitry described above arranged into a parallel processing configuration. For instance, a set of graphical processing units (e.g., GPUs from the GeForce®, Quadro®, Tesla® family manufactured by Nvidia™, GMA, Iris™ family manufactured by Intel™, or the Radeon™ family manufactured by AMD™) may be arranged to perform highly parallel or repetitive computing tasks simultaneously. Other specialized parallel processing units or hardware capable of performing multiple calculations simultaneously may also be deployed. GPUs may include a single GPU “device” or “system” which operates or orchestrates numerous (e.g., tens, hundreds, or thousands) of sub-processors; many of the examples provided herein refer to the use of a single GPU device or system which uses each of its numerous sub-processors to process a respective set of parameters.

Although not depicted, the radiotherapy processing computing system 110 may include a communication interface, network interface card, and communications circuitry. An example communication interface may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as an IEEE 802.11/Wi-Fi adapter), a telecommunication adapter (e.g., to communicate with 3G, 4G/LTE, and 5G networks and the like), and the like. Such a communication interface may include one or more digital and/or analog communication devices that permit a machine to communicate with other machines and devices, such as remotely located components, via a network. The network may provide the functionality of a local area network (LAN), a wireless network, a cloud computing environment (e.g., software as a service, platform as a service, infrastructure as a service, etc.), a client-server, a wide area network (WAN), and the like. For example, the network may be a LAN or a WAN that may include other systems (including additional image processing computing systems or image-based components associated with medical imaging or radiotherapy operations).

In an example, the radiotherapy processing computing system 110 may obtain image data 152 from the image data source 150 (e.g., MR images) for hosting on the storage device 116 and the memory 114. In yet another example, the software programs may substitute functions of the patient images such as signed distance functions or processed versions of the images that emphasize some aspect of the image information. The radiotherapy processing computing system 110 may obtain or communicate image data 152 from or to image data source 150. In further examples, the treatment data source 160 receives or updates the planning data 162 as a result of a treatment plan generated by the radiotherapy planning logic 120. The image data source 150 may also provide or host the imaging data for use in the radiotherapy planning logic 120.

In an example, computing system 110 may communicate with treatment data source(s) 160, input device 148, and other data sources to generate optimization variables and parameters for a plurality of radiotherapy treatment plan optimization problems. Such optimization variables and parameters are generated to identify a plurality of solutions to the radiotherapy problem. These solutions may approximate a solution, and may be further evaluated and refined before use (e.g., with additional optimization) in a radiotherapy treatment.

The processing circuitry 112 and the parallel processing circuitry 118 may be communicatively coupled to the memory 114 and the storage device 116, and the processing circuitry 112 and the parallel processing circuitry 118 may be configured to execute computer-executable instructions stored thereon from either the memory 114 or the storage device 116. Particularly, radiotherapy planning logic 120 receives an optimization problem that is derived from parameters for radiotherapy treatment. The processing circuitry 112 and parallel processing circuitry 118 may utilize software programs or implementations to optimize a radiotherapy dose for delivery to a patient, as part of developing an optimized solution to a radiotherapy problem as discussed herein. Further, such software programs or implementations may utilize the radiotherapy planning logic 120 to produce new or updated treatment plan parameters for deployment to the treatment data source 160 and/or presentation on output device 146, using the techniques further discussed herein. The processing circuitry 112 or the parallel processing circuitry 118 may subsequently then transmit the new or updated treatment plan details via a communication interface and the network to the treatment device 180, where the radiation therapy plan will be used to treat a patient with radiation via the treatment device 180, consistent with results of the radiotherapy planning logic 120 (e.g., according to the processes discussed below).

In an example, the image data 152 used for defining a radiotherapy problem or indicating the anatomical areas of the patient may include one or more MRI image (e.g., 2D MRI, 3D MRI, 2D streaming MRI, 4D MRI, 4D volumetric MRI, 4D cine MRI, etc.), functional MRI images (e.g., fMRI, DCE-MRI, diffusion MRI), Computed Tomography (CT) images (e.g., 2D CT, 2D Cone beam CT, 3D CT, 3D CBCT, 4D CT, 4DCBCT), ultrasound images (e.g., 2D ultrasound, 3D ultrasound, 4D ultrasound), Positron Emission Tomography (PET) images, X-ray images, fluoroscopic images, radiotherapy portal images, Single-Photo Emission Computed Tomography (SPECT) images, computer-generated synthetic images (e.g., pseudo-CT images) and the like. Further, the image data 152 may also include or be associated with medical image processing data (for example, training images, ground truth images, contoured images, and dose images). In other examples, an equivalent representation of an anatomical area may be represented in non-image formats (e.g., coordinates, mappings, etc.).

In an example, the image data 152 may be received from the image acquisition device 170 and stored in one or more of the image data sources 150 (e.g., a Picture Archiving and Communication System (PACS), a Vendor Neutral Archive (VNA), a medical record or information system, a data warehouse, etc.). Accordingly, the image acquisition device 170 may comprise an MRI imaging device, a CT imaging device, a PET imaging device, an ultrasound imaging device, a fluoroscopic device, a SPECT imaging device, an integrated Linear Accelerator and MRI imaging device, CBCT imaging device, or other medical imaging devices for obtaining the medical images of the patient. The image data 152 may be received and stored in any type of data or any type of format (e.g., in a Digital Imaging and Communications in Medicine (DICOM) format) that the image acquisition device 170 and the radiotherapy processing computing system 110 may use to perform operations consistent with the disclosed embodiments. Further, in some examples, the models discussed herein may be trained to process the original image data format or a derivation thereof.

In an example, the image acquisition device 170 may be integrated with the treatment device 180 as a single apparatus (e.g., an MRI device combined with a linear accelerator, also referred to as an “MRI-Linac”). Such an MRI-Linac can be used, for example, to determine a location of a target organ or a target tumor in the patient so as to direct radiation therapy accurately according to the radiation therapy treatment plan to a predetermined target. For instance, a radiation therapy treatment plan may provide information about a particular radiation dose to be applied to each patient. The radiation therapy treatment plan may also include other radiotherapy information, including control points of a radiotherapy treatment device, such as couch position, beam intensity, beam angles, dose-histogram-volume information, the number of radiation beams to be used during therapy, the dose per beam, and the like.

The radiotherapy processing computing system 110 may communicate with an external database through a network to send/receive a plurality of various types of data related to image processing and radiotherapy operations. For example, an external database may include machine data (including device constraints) that provides information associated with the treatment device 180, the image acquisition device 170, or other machines relevant to radiotherapy or medical procedures. Machine data information (e.g., control points) may include radiation beam size, arc placement, beam on and off time duration, machine parameters, segments, multi-leaf collimator (MLC) configuration, gantry speed, MRI pulse sequence, and the like. The external database may be a storage device and may be equipped with appropriate database administration software programs. Further, such databases or data sources may include a plurality of devices or systems located either in a central or a distributed manner.

The radiotherapy processing computing system 110 can collect and obtain data, and communicate with other systems, via a network using one or more communication interfaces, which are communicatively coupled to the processing circuitry 112 and the memory 114. For instance, a communication interface may provide communication connections between the radiotherapy processing computing system 110 and radiotherapy system components (e.g., permitting the exchange of data with external devices). For instance, the communication interface may, in some examples, have appropriate interfacing circuitry from an output device 146 or an input device 148 to connect to the user interface 142, which may be a hardware keyboard, a keypad, or a touch screen through which a user may input information into the radiotherapy system.

As an example, the output device 146 may include a display device that outputs a representation of the user interface 142 and one or more aspects, visualizations, or representations of the medical images, the treatment plans, and statuses of training, generation, verification, or implementation of such plans. The output device 146 may include one or more display screens that display medical images, interface information, treatment planning parameters (e.g., contours, dosages, beam angles, labels, maps, etc.), treatment plans, a target, localizing a target and/or tracking a target, or any related information to the user. The input device 148 connected to the user interface 142 may be a keyboard, a keypad, a touch screen or any type of device that a user may use to the radiotherapy system 100. Alternatively, the output device 146, the input device 148, and features of the user interface 142 may be integrated into a single device such as a smartphone or tablet computer (e.g., Apple iPad®, Lenovo Thinkpad®, Samsung Galaxy®, etc.).

Furthermore, any and all components of the radiotherapy system may be implemented as a virtual machine (e.g., via VMWare, Hyper-V, and the like virtualization platforms) or independent devices. For instance, a virtual machine can be software that functions as hardware. Therefore, a virtual machine can include at least one or more virtual processors, one or more virtual memories, and one or more virtual communication interfaces that together function as hardware. For example, the radiotherapy processing computing system 110, the image data sources 150, or like components, may be implemented as a virtual machine or within a cloud-based virtualization environment.

The image acquisition device 170 can be configured to acquire one or more images of the patient's anatomy for a region of interest (e.g., a target organ, a target tumor or both). Each image, typically a 2D image or slice, can include one or more parameters (e.g., a 2D slice thickness, an orientation, and a location, etc.). In an example, the image acquisition device 170 can acquire a 2D slice in any orientation. For example, an orientation of the 2D slice can include a sagittal orientation, a coronal orientation, or an axial orientation. The processing circuitry 112 can adjust one or more parameters, such as the thickness and/or orientation of the 2D slice, to include the target organ and/or target tumor. In an example, 2D slices can be determined from information such as a 3D CBCT or CT or MRI volume. Such 2D slices can be acquired by the image acquisition device 170 in “near real time” while a patient is undergoing radiation therapy treatment (for example, when using the treatment device 180 (with “near real time” meaning acquiring the data in at least milliseconds or less)).

The radiotherapy planning logic 120 in the radiotherapy processing computing system 110 implements a radiotherapy optimization workflow 130 and treatment plan generation workflow 140, The radiotherapy optimization workflow 130 may implement optimization operations for identifying and developing radiotherapy plans, while the treatment plan generation workflow may implement operations for evaluating, selecting, and refining one of the radiotherapy plans. In specific examples, the radiotherapy optimization workflow 130 performs radiotherapy problem processing 132 to obtain and identify an optimization problem, problem conversion processing 134 to convert optimization problems for more effective or efficient execution on hardware (such as converting the optimization problems for execution with the parallel processing circuitry 118), and solution processing 136 to identify and output solutions to the optimization problems. More details of the radiotherapy optimization workflow 130 are provided below with reference to FIGS. 5 and 6, including with the use of an alternating direction method of multipliers to help convert equations for efficient execution on the parallel processing circuitry 118. Likewise, more details of the treatment plan generation workflow 140 are provided below with reference to FIGS. 5 and 7, which indicate how a treatment plan may be further evaluated, selected, and optimized, using a found solution as a starting point for such a treatment plan.

FIG. 2A illustrates a radiation therapy device 202 that may include a radiation source, such as an X-ray source or a linear accelerator, a couch 216, an imaging detector 214, and a radiation therapy output 204. The radiation therapy device 202 may be configured to emit a radiation beam 208 to provide therapy to a patient. The radiation therapy output 204 can include one or more attenuators or collimators, such as an MLC. An MLC may be used for shaping, directing, or modulating an intensity of a radiation therapy beam to the specified target locus within the patient. The leaves of the MLC, for instance, can be automatically positioned to define an aperture approximating a tumor cross-section or projection, and cause modulation of the radiation therapy beam. For example, the leaves can include metallic plates, such as comprising tungsten, with a long axis of the plates oriented parallel to a beam direction and having ends oriented orthogonally to the beam direction. Further, a “state” of the MLC can be adjusted adaptively during a course of radiation therapy treatment, such as to establish a therapy beam that better approximates a shape or location of the tumor or other target locus.

Referring back to FIG. 2A, a patient can be positioned in a region 212 and supported by the treatment couch 216 to receive a radiation therapy dose, according to a radiation therapy treatment plan. The radiation therapy output 204 can be mounted or attached to a gantry 206 or other mechanical support. One or more chassis motors (not shown) may rotate the gantry 206 and the radiation therapy output 204 around couch 216 when the couch 216 is inserted into the treatment area. In an example, gantry 206 may be continuously rotatable around couch 216 when the couch 216 is inserted into the treatment area. In another example, gantry 206 may rotate to a predetermined position when the couch 216 is inserted into the treatment area. For example, the gantry 206 can be configured to rotate the therapy output 204 around an axis (“A”). Both the couch 216 and the radiation therapy output 204 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 216 movements or rotations in order to properly position the patient in or out of the radiation beam 208 according to a radiation therapy treatment plan. Both the couch 216 and the gantry 206 are independently moveable from one another in multiple degrees of freedom, which allows the patient to be positioned such that the radiation beam 208 can target the tumor precisely. The MLC may be integrated and included within gantry 206 to deliver the radiation beam 208 of a certain shape.

The coordinate system (including axes A, T, and L) shown in FIG. 2A can have an origin located at an isocenter 210. The isocenter can be defined as a location where the central axis of the radiation beam 208 intersects the origin of a coordinate axis, such as to deliver a prescribed radiation dose to a location on or within a patient. Alternatively, the isocenter 210 can be defined as a location where the central axis of the radiation beam 208 intersects the patient for various rotational positions of the radiation therapy output 204 as positioned by the gantry 206 around the axis A. As discussed herein, the gantry angle corresponds to the position of gantry 206 relative to axis A, although any other axis or combination of axes can be referenced and used to determine the gantry angle.

Gantry 206 may also have an attached imaging detector 214. The imaging detector 214 is preferably located opposite to the radiation source, and in an example, the imaging detector 214 can be located within a field of the radiation beam 208. The imaging detector 214 can be mounted on the gantry 206 (preferably opposite the radiation therapy output 204), such as to maintain alignment with the radiation beam 208. The imaging detector 214 rotates about the rotational axis as the gantry 206 rotates. In an example, the imaging detector 214 can be a flat panel detector (e.g., a direct detector or a scintillator detector). In this manner, the imaging detector 214 can be used to monitor the radiation beam 208 or the imaging detector 214 can be used for imaging the patient's anatomy, such as portal imaging. The control circuitry of the radiation therapy device 202 may be integrated within the radiotherapy system 100 or remote from it.

In an illustrative example, one or more of the couch 216, the therapy output 204, or the gantry 206 can be automatically positioned, and the therapy output 204 can establish the radiation beam 208 according to a specified dose for a particular therapy delivery instance. A sequence of therapy deliveries can be specified according to a radiation therapy treatment plan, such as using one or more different orientations or locations of the gantry 206, couch 216, or therapy output 204. The therapy deliveries can occur sequentially, but can intersect in a desired therapy locus on or within the patient, such as at the isocenter 210. A prescribed cumulative dose of radiation therapy can thereby be delivered to the therapy locus while damage to tissue near the therapy locus can be reduced or avoided.

FIG. 2B illustrates a radiation therapy device 202 that may include a combined Linac and an imaging system, such as a CT imaging system. The radiation therapy device 202 can include an MLC (not shown). The CT imaging system can include an imaging X-ray source 218, such as providing X-ray energy in a kiloelectron-Volt (keV) energy range. The imaging X-ray source 218 can provide a fan-shaped and/or a conical radiation beam 208 directed to an imaging detector 222, such as a flat panel detector. The radiation therapy device 202 can be similar to the system described in relation to FIG. 2A, such as including a radiation therapy output 204, a gantry 206, a couch 216, and another imaging detector 214 (such as a flat panel detector). The X-ray source 218 can provide a comparatively-lower-energy X-ray diagnostic beam, for imaging.

In the illustrative example of FIG. 2B, the radiation therapy output 204 and the X-ray source 218 can be mounted on the same rotating gantry 206, rotationally separated from each other by 90 degrees. In another example, two or more X-ray sources can be mounted along the circumference of the gantry 206, such as each having its own detector arrangement to provide multiple angles of diagnostic imaging concurrently. Similarly, multiple radiation therapy outputs 204 can be provided.

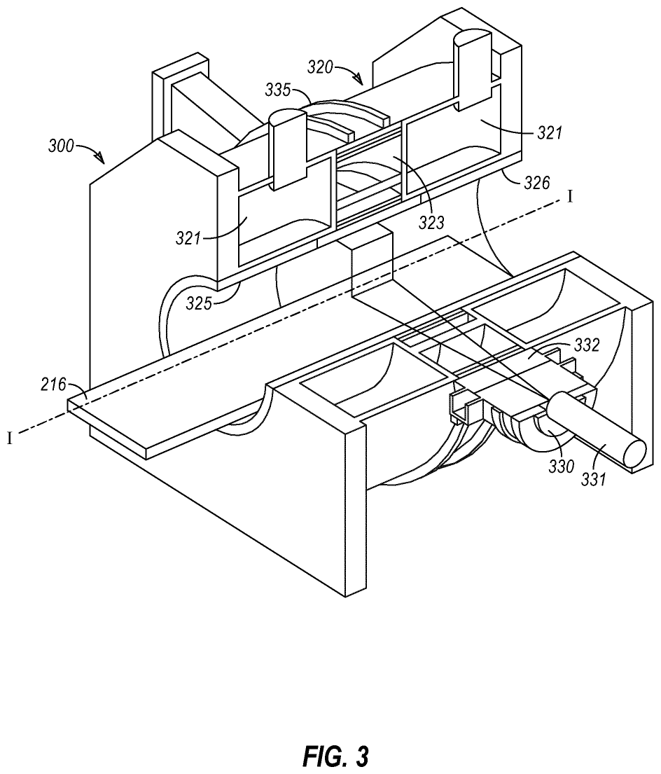

FIG. 3 depicts a radiation therapy system 300 that can include combining a radiation therapy device 202 and an imaging system, such as a magnetic resonance (MR) imaging system (e.g., known in the art as an MR-LINAC) consistent with the disclosed examples. As shown, system 300 may include a couch 216, an image acquisition device 320, and a radiation delivery device 330. System 300 delivers radiation therapy to a patient in accordance with a radiotherapy treatment plan. In some examples, image acquisition device 320 may correspond to image acquisition device 170 in FIG. 1 that may acquire origin images of a first modality (e.g., an MRI image) or destination images of a second modality (e.g., an CT image).

Couch 216 may support a patient (not shown) during a treatment session. In some implementations, couch 216 may move along a horizontal translation axis (labelled “I”), such that couch 216 can move the patient resting on couch 216 into and/or out of system 300. Couch 216 may also rotate around a central vertical axis of rotation, transverse to the translation axis. To allow such movement or rotation, couch 216 may have motors (not shown) enabling the couch 216 to move in various directions and to rotate along various axes. A controller (not shown) may control these movements or rotations in order to properly position the patient according to a treatment plan.

In some examples, image acquisition device 320 may include an MRI machine used to acquire 2D or 3D MRI images of the patient before, during, and/or after a treatment session. Image acquisition device 320 may include a magnet 321 for generating a primary magnetic field for magnetic resonance imaging. The magnetic field lines generated by operation of magnet 321 may run substantially parallel to the central translation axis I. Magnet 321 may include one or more coils with an axis that runs parallel to the translation axis I. In some examples, the one or more coils in magnet 321 may be spaced such that a central window 323 of magnet 321 is free of coils. In other examples, the coils in magnet 321 may be thin enough or of a reduced density such that they are substantially transparent to radiation of the wavelength generated by radiotherapy device 330. Image acquisition device 320 may also include one or more shielding coils, which may generate a magnetic field outside magnet 321 of approximately equal magnitude and opposite polarity in order to cancel or reduce any magnetic field outside of magnet 321. As described below, radiation source 331 of radiation delivery device 330 may be positioned in the region where the magnetic field is cancelled, at least to a first order, or reduced.

Image acquisition device 320 may also include two gradient coils 325 and 326, which may generate a gradient magnetic field that is superposed on the primary magnetic field. Coils 325 and 326 may generate a gradient in the resultant magnetic field that allows spatial encoding of the protons so that their position can be determined. Gradient coils 325 and 326 may be positioned around a common central axis with the magnet 321 and may be displaced along that central axis. The displacement may create a gap, or window, between coils 325 and 326. In examples where magnet 321 can also include a central window 323 between coils, the two windows may be aligned with each other.

In some examples, image acquisition device 320 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 320 concerns certain examples and is not intended to be limiting.

Radiation delivery device 330 may include the radiation source 331, such as an X-ray source or a Linac, and an MLC 332. Radiation delivery device 330 may be mounted on a chassis 335. One or more chassis motors (not shown) may rotate the chassis 335 around the couch 216 when the couch 216 is inserted into the treatment area. In an example, the chassis 335 may be continuously rotatable around the couch 216, when the couch 216 is inserted into the treatment area. Chassis 335 may also have an attached radiation detector (not shown), preferably located opposite to radiation source 331 and with the rotational axis of the chassis 335 positioned between the radiation source 331 and the detector. Further, the device 330 may include control circuitry (not shown) used to control, for example, one or more of the couch 216, image acquisition device 320, and radiotherapy device 330. The control circuitry of the radiation delivery device 330 may be integrated within the system 300 or remote from it.

During a radiotherapy treatment session, a patient may be positioned on couch 216. System 300 may then move couch 216 into the treatment area defined by the magnet 321, coils 325, 326, and chassis 335. Control circuitry may then control radiation source 331, MLC 332, and the chassis motor(s) to deliver radiation to the patient through the window between coils 325 and 326 according to a radiotherapy treatment plan.

FIG. 2A, FIG. 2B, and FIG. 3 generally illustrate examples of a radiation therapy device configured to provide radiotherapy treatment to a patient, using a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another example, the therapy output can be fixed, such as located in a region laterally separated from the patient, and a platform supporting the patient can be used to align a radiation therapy isocenter with a specified target locus within the patient.

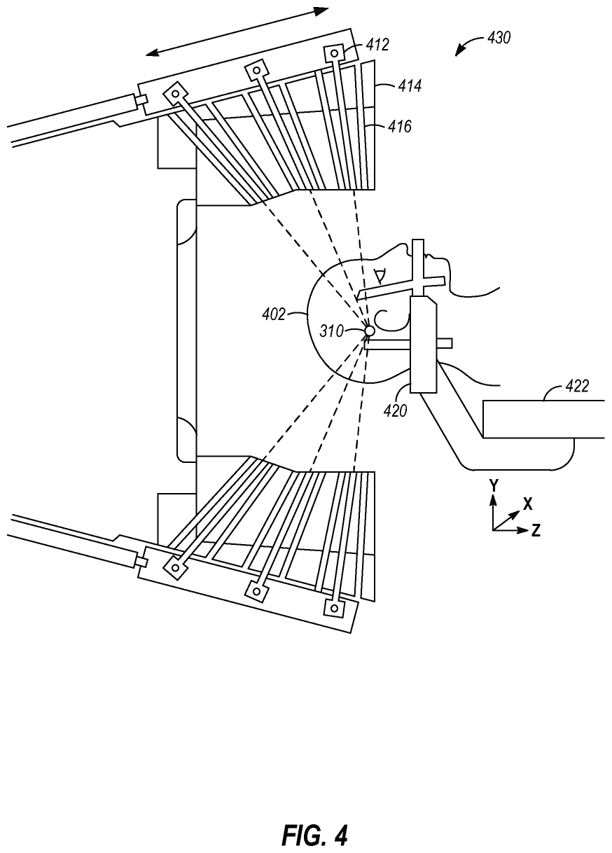

FIG. 4 illustrates a contrasting example of a Leksell Gamma Knife radiotherapy device 430, which provides such radiotherapy treatment by means of gamma radiation. As a brief overview of a Gamma Knife device, radiation is emitted from a large number of fixed radioactive sources and is focused by means of collima-tors, i.e. passages or channels for obtaining a beam of limited cross section, towards a defined target or treatment volume. Each of the sources provides a dose of gamma radiation which is insufficient to damage intervening tissue. However, tissue destruction occurs where the radiation beams from all or some radiation sources intersect or converge, causing the radiation to reach tissue-destructive levels. The point of convergence is hereinafter referred to as the “isocenter” but may also be referred to as a “focus point”.

As shown in FIG. 4, in a radiotherapy treatment session, a patient 402 may wear a coordinate frame 420 to keep stable the patient's body part (e.g., the head) undergoing surgery or radiotherapy. Coordinate frame 420 and a patient positioning system 422 may establish a spatial coordinate system, which may be used while imaging a patient or during radiation surgery. Radiotherapy device 430 may include a protective housing 414 to enclose a plurality of radiation sources 412. Radiation sources 412 may generate a plurality of radiation beams (e.g., beamlets) through beam channels 416. The plurality of radiation beams may be configured to focus on an isocenter 310 from different directions. While each individual radiation beam may have a relatively low intensity, isocenter 310 may receive a relatively high level of radiation when multiple doses from different radiation beams accumulate at isocenter 310. In certain examples, isocenter 310 may correspond to a target under surgery or treatment, such as a tumor.

Other types of radiotherapy devices (not illustrated) use protons and/or ions to deliver the radiotherapy treatment. The direction and shape of the radiation beam should be accurately controlled to ensure that the tumor receives the prescribed radiation dose, and the radiation from the beam should minimize damage to the surrounding healthy tissue, especially the organ(s) at risk (OARs). Thus, treatment planning is used to design and control radiation beam parameters, and a radiotherapy device effectuates a treatment by delivering a spatially varying dose distribution to the patient.

Treatment plan optimization for radiation therapy, such as for Gamma knife radiosurgery, aims at maximizing the dose delivered to the target volume within the patient (e.g. in treatment of tumors) at the same time as the dose delivered to adjacent normal tissues is minimized. In treatment plan optimization, the delivered radiation dose is mainly limited by two competing factors: the first one is delivering a high dose to the target volume and the second one is delivering low dose to the surrounding normal tissues. The treatment plan optimization is a process including optimizing the number of shots being used, the sector-collimator combinations, the shot times, and the position of the shot (i.e. isocenter). Clearly, the irregularity and size of a target volume greatly influence the number of shots needed and the size of the shots being used to optimize the treatment. Thus, for gamma knife radiotherapy, the selected isocenter locations and their corresponding shots for a given case constitutes a treatment plan.

FIG. 5 provides a high-level view of radiotherapy treatment planning workflow operations. Specifically, this workflow uses parallel processing for radiotherapy problem optimization, that can generate all or nearly all Pareto-optimal solutions for a radiotherapy problem.

The operations in FIG. 5, in more detail, illustrate how radiotherapy problem information 510 for a treatment human subject are provided with the definition of information such as target areas of treatment 512 and organ at risk areas 514. Other information relevant to the radiotherapy problem may include radiotherapy machine information 520 such as machine capabilities 522.

A suitable dose distribution to be delivered with radiotherapy problem is then optimized and solved with the depicted radiotherapy problem optimization 530. Such optimization and solution may occur with the use of parallel processing hardware 535, and the transformation or modification of the radiotherapy problems for use with the parallel processing hardware. As discussed, the parallel processing hardware may include a plurality of graphics processing units (GPUs), and the relevant transformation of the problem may include the use of an alternating direction method of multipliers technique to solve linear programming equations. The alternating direction method of multipliers may transform the linear programming equations into matrix and projection operations.

The optimization 530 produces a plurality of Pareto-optimal radiotherapy solutions 540, which may consist of a Pareto surface or frontier of all (or approximately all) available pareto-optimized solutions. One or more of these solutions may be selected and refined with operation 550, and used for generation of a particular radiotherapy treatment plan with operation 560. The generation of the radiotherapy treatment plan with operation 560 (and, related selection or modification of the radiotherapy solution) may be dependent on machine capabilities 522. Finally, radiotherapy treatment may be delivered using the generated treatment plan.

FIG. 6 provides an example workflow for applying an alternating direction method of multipliers to parallelize execution of linear or quadradic programming equations. In an example, the alternating direction method of multipliers (ADMM) follows the algorithmic approach discussed in Boyd et al., Distributed Optimization and Statistical Learning via the Alternating Direction Method of Multipliers, Now Publishers Inc. (2011), and applied as a batch LP solver in Nair et al., Solving Mixed Integer Programs Using Neural Networks, arXiv: 2012.13349 (2020), both of which are incorporated by reference in their entirety.

The ADMM algorithm solves convex optimization problems by breaking them into smaller pieces, each of which are then easier to handle. The ADMM is well suited for use as a batch LP solver. In an example, the optimization problem can be expressed as:

$$\begin{matrix}minimize f ( x ) + g ( z ) \\ subject to x = z\end{matrix} \qquad ( Equation 3 )$$

With this optimization problem in mind, the ADMM solver can be expressed as:

for any ρ<0:

$$\begin{matrix}{x}^{k + 1} = arg {min}_{x} ( f ( x ) + ( ρ / 2 ) {‖ x - {z}^{k} - {λ}^{k} ‖}_{2}^{2} ) \\ {z}^{k + 1} = arg \vert {min}_{z} ( g ( z ) + ( ρ / 2 ) {‖ {x}^{k + 1} - z - {λ}^{k} ‖}_{2}^{2} ) \\ {λ}^{k + 1} = {λ}^{k} + {x}^{k + 1} - {z}^{k + 1}\end{matrix} . \qquad ( Equation 4 )$$

For a LP problem this can be re-formulated as

$$\begin{matrix}{x}^{k + 1} = K ( {z}^{k} + {λ}^{k} ) + {Γ}_{ρ} \\ {z}^{k + 1} = {\prod }_{w} ( {x}^{k + 1} - {λ}^{k} ) \\ {λ}^{k + 1} = {λ}^{k} - ( {x}^{k + 1} - {z}^{k + 1} )\end{matrix} \qquad ( Equation 5 )$$

With this formulation,

$$\begin{matrix}Q = {( \begin{matrix}1 & {A}^{T} \\ A & - 1\end{matrix} )}^{- 1} , P = ( \begin{matrix}0 & 0 \\ 0 & 1\end{matrix} ) K = Q + P ; \\ {Γ}_{ρ} Q ( \begin{matrix}- {c}_{ρ} \\ 0\end{matrix} ) , {c}_{ρ} = \frac{c}{ρ}\end{matrix} \qquad ( Equation 6 )$$

Where A is the constraint matrix and c is the linear cost function vector. π_{w} is an operator that projects points onto the feasible set and is dependent on the weights multiplying the objective function terms in the objection function. It projects each element in a vector to a closed or half-open interval. Thus, each optimization is carried out with its unique projection operator which is quickly calculated. The operator K is, however, common for all optimizations but more time demanding to calculate.

In an example, the use of the ADMM algorithm to solve a radiotherapy problem, may be summarized with the following operations, depicted in FIG. 6:

Calculate the K-operator (operation 601) and represent it, for example, explicitly or in factorized form.

Transfer the K-operator to each node (operation 602).

Run the iteration scheme (operation 603), such as described with reference to Equation 5, until the difference in variables between two successive iterations is less than a pre-defined threshold (operation 604). In an example, the iteration scheme involves matrix multiplications and a projection.

The ADMM formulation can provide an equally accurate solution as other solution methods but may require longer time for processing if particularly accurate solutions are desirable. However, it will be understood that ADMM provides a reasonably exact solution after a few iterations, useful for Pareto navigation. Such a chosen plan can then be improved upon by other optimization methods (Simplex, interior point, ADMM, . . . ).

FIG. 7 illustrates a flowchart 700 of a method of radiotherapy treatment planning based on the techniques discussed above. For instance, the following features of flowchart 700 may be integrated or adapted with the optimization operations discussed with reference to FIG. 5, and optimization solver operations discussed with reference to FIG. 6.

Operation 710 begins with operations to obtain a radiotherapy problem, with the radiotherapy problem defining various parameters for delivery of a radiotherapy treatment from a radiotherapy machine. Such a radiotherapy problem may be adjustable via parameters, which are optionally received with a request to solve the radiotherapy problem.

Operation 720 proceeds with operations to perform a treatment plan optimization for a radiotherapy problem, which may be repeated for individual problems until an entire Pareto surface or frontier (or, a sufficient portion of the pareto surface or frontier) of solutions are identified. Although not shown, the treatment plan optimization operations may be performed in parallel or concurrently. The treatment plan optimization may be based on dose, geometry, imaging, machine learning, radiobiology, or other relevant factors.

Operation 730 proceeds to identify linear programming equations from the radiotherapy problem. At operation 740, these linear programming equations are converted (e.g., changed, transformed, etc.) for execution on parallel processing hardware, such as with use of the alternating direction method of multipliers algorithm discussed above. Then, at operation 750, multiple linear programming equations are solved in parallel on the parallel processing hardware.

At operation 760, a Pareto surface or frontier of Pareto-optimal radiotherapy solutions are identified. This set of solutions may be used to provide a particular solution for use in radiotherapy treatment, in operation 770. At operation 770, one of the plurality of pareto-optimal solutions is selected, evaluated, and potentially improved. Finally, at operation 780, a treatment plan is generated based on the optimized (pareto-optimal) radiotherapy solution.

Although not directly depicted in the drawings, various forms of user interfaces or representations may be provided to enable a representation of a solution space to the radiotherapy problem, based on the plurality of pareto-optimal solutions. This may be provided in a graphical user interface having functionality to configure the treatment plan, and to receive and output data related to the treatment plan. For instance, information associated with a solution to the radiotherapy problem, for a particular set of parameters, may be displayed. User interaction may be obtained in such a user interface for modifying the particular set of parameters. As will be understood, a variety of interactive Pareto navigation functions may be provided, such as an approximation of the Pareto surface that lets a user explore estimates of solutions corresponding to new or different parameter values that have not been evaluated.

It will be understood that the Pareto optimal plans generated with such techniques can be considered as points on a Pareto surface. The more points that are generated, the better the description of the surface. When a user explores the Pareto surface, the user performs some type of interpolation between existing calculated points in order to select and use a solution. Plausibly, the user will ultimately develop and choose a plan that is not necessarily one of the calculated points.

Accordingly, in some examples, all the Pareto-optimal solutions, or at least a substantial subset of the pareto-optimal solutions, may be generated and evaluated before a particular plan or solution is selected by a user. Thus, the ultimately chosen plan may be a plan “in between” two generated points on the pareto-surface. Because all the points on the Pareto surface can be generated using the ADMM techniques discussed herein, navigating the Pareto surface can be performed quickly and efficiently.

FIG. 8 illustrates a block diagram of an example of a machine 800 on which one or more of the methods as discussed herein can be implemented. In one or more examples, one or more items of the radiotherapy processing computing system 110 can be implemented by the machine 800. In alternative examples, the machine 800 operates as a standalone device or may be connected (e.g., networked) to other machines. In one or more examples, the radiotherapy processing computing system 110 can include one or more of the items of the machine 800. In a networked deployment, the machine 800 may operate in the capacity of a server or a client machine in server-client network environment, or as a peer machine in a peer-to-peer (or distributed) network environment. The machine may be a personal computer (PC), server, a tablet, smartphone, a web appliance, edge computing device, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example machine 800 includes processing circuitry or processor 802 (e.g., a CPU, a graphics processing unit (GPU), an ASIC, circuitry, such as one or more transistors, resistors, capacitors, inductors, diodes, logic gates, multiplexers, buffers, modulators, demodulators, radios (e.g., transmit or receive radios or transceivers), sensors 821 (e.g., a transducer that converts one form of energy (e.g., light, heat, electrical, mechanical, or other energy) to another form of energy), or the like, or a combination thereof), a main memory 804 and a static memory 806, which communicate with each other via a bus 808. The machine 800 (e.g., computer system) may further include a video display device 810 (e.g., a liquid crystal display (LCD) or a cathode ray tube (CRT)). The machine 800 also includes an alphanumeric input device 812 (e.g., a keyboard), a user interface (UI) navigation device 814 (e.g., a mouse), a disk drive or mass storage unit 816, a signal generation device 818 (e.g., a speaker), and a network interface device 820.

The disk drive unit 816 includes a machine-readable medium 822 on which is stored one or more sets of instructions and data structures (e.g., software) 824 embodying or utilized by any one or more of the methodologies or functions described herein. The instructions 824 may also reside, completely or at least partially, within the main memory 804 and/or within the processor 802 during execution thereof by the machine 800, the main memory 804 and the processor 802 also constituting machine-readable media.

The machine 800 as illustrated includes an output controller 828. The output controller 828 manages data flow to/from the machine 800. The output controller 828 is sometimes called a device controller, with software that directly interacts with the output controller 828 being called a device driver.

While the machine-readable medium 822 is shown in an example to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present disclosure, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), EEPROM, and flash memory devices; magnetic disks such as internal hard disks and removable disks; magneto-optical disks; and CD-ROM and DVD-ROM disks.

The instructions 824 may further be transmitted or received over a communications network 826 using a transmission medium. The instructions 824 may be transmitted using the network interface device 820 and any one of a number of well-known transfer protocols (e.g., HTTP). Examples of communication networks include a LAN, a WAN, the Internet, mobile telephone networks, Plain Old Telephone (POTS) networks, and wireless data networks (e.g., Wi-Fi and 4G/5G data networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples can include a computer-readable medium or machine-readable medium encoded with instructions operable to configure an electronic device to perform methods as described in the above examples. An implementation of such methods can include software code, such as microcode, assembly language code, a higher-level language code, or the like (e.g., “source code”). Such software code can include computer-readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., read only memories (ROMs)), EPROMS, EEPROMS, or any type of media suitable for storing electronic instructions, and the like. Such computer-readable storage medium is coupled to a computer system bus to be accessible by the processor and other parts of the OIS.

In an embodiment, the computer-readable storage medium may have encoded a data structure for treatment planning, wherein the treatment plan may be adaptive. The data structure for the computer-readable storage medium may be at least one of a Digital Imaging and Communications in Medicine (DICOM) format, an extended DICOM format, an XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, C, C++, C#, Java, Python, CUDA programming, and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

1. A computer-implemented method for radiotherapy treatment planning, the method comprising:
obtaining a radiotherapy problem for providing radiotherapy treatment to a human subject from a radiotherapy treatment machine, the radiotherapy problem being a multicriteria optimization problem that is adjustable via a plurality of parameters;
performing treatment planning optimization for delivery of the radiotherapy treatment, the treatment planning optimization comprising:
identifying parameterized linear programming equations from the radiotherapy problem;
converting the parameterized linear programming equations for execution by parallel processing hardware; and
solving a plurality of the converted parameterized linear programming equations in parallel on the parallel processing hardware, to produce a plurality of solutions to the radiotherapy problem corresponding to the plurality of parameters that define the radiotherapy problem; and
generating treatment plan data based on at least one of the plurality of solutions, wherein the treatment plan data is used to generate a radiation therapy treatment plan.

2. The method of claim 1, wherein the parameters defined by the radiotherapy problem correspond to clinical preferences.

3. The method of claim 1, further comprising:
receiving a plurality of sets of the parameters that define the radiotherapy problem.

4. The method of claim 3, wherein a parameter in the plurality of sets of the parameters concerns a particular anatomical area of the human subject to receive the radiotherapy treatment from the radiotherapy treatment machine.

5. The method of claim 3, wherein the plurality of sets of the parameters include definitions of one or more organ at risk areas and one or more target areas.

6. The method of claim 1, wherein converting the parameterized linear programming equations comprises applying an alternating direction method of multipliers technique.

7. The method of claim 6, wherein the alternating direction method of multipliers technique comprises transforming the converted parameterized linear programming equations to matrix operations.

8. The method of claim 1, wherein the parallel processing hardware comprises a plurality of graphics processing units (GPUs).

9. The method of claim 1, further comprising generating a representation of a solution space based on the plurality of solutions to the radiotherapy problem, wherein the solution space is a Pareto surface comprising a set of Pareto optimal solutions.

10. The method of claim 1, further comprising:
generating a display of a graphical user interface, the graphical user interface configured to provide functionality to configure the radiation therapy treatment plan;
displaying, within the graphical user interface, information associated with a particular solution to the radiotherapy problem for a particular set of the parameters; and
obtaining user interaction with the graphical user interface to modify the particular set of parameters.

11. The method of claim 1, further comprising:
selecting a solution to the radiotherapy problem based on evaluation of the plurality of solutions;
wherein the treatment plan data is generated based on the selected solution to the radiotherapy problem.

12. The method of claim 11, wherein the selected solution to the radiotherapy problem provides an approximate solution, with the method further comprising:
receiving an additional optimization to the selected solution;
wherein the treatment plan data is generated based on the additional optimization to the selected solution.

13. The method of claim 1, wherein the treatment plan data for the radiotherapy treatment comprises a set of treatment delivery parameters corresponding to capabilities of the radiotherapy treatment machine.

14. The method of claim 13, wherein the radiotherapy treatment is to be provided with a Gamma knife, and wherein the set of treatment delivery parameters comprises a set of isocenters used for delivery of the radiotherapy treatment.

15. The method of claim 14, wherein the set of treatment delivery parameters further comprises timing for delivery of the radiotherapy treatment and a collimator sequence for the delivery of the radiotherapy treatment.

16. The method of claim 13, wherein the radiotherapy treatment is to be provided with a Linac or magnetic resonance (MR)-Linac radiotherapy machine.

17. The method of claim 16, wherein the radiotherapy treatment is to be provided with Volumetric-modulated arc therapy (VMAT) or Intensity modulated radiation therapy (IMRT), and wherein the set of treatment delivery parameters comprises: a set of arc control points for one or more arcs, fluence fields, gantry speed, and dose rate along the one or more arcs.

18. A non-transitory computer-readable storage medium comprising computer-readable instructions for radiotherapy treatment planning, wherein the instructions, when executed with a computing machine, cause the computing machine to:
obtain a radiotherapy problem for providing radiotherapy treatment to a human subject from a radiotherapy treatment machine, the radiotherapy problem being a multicriteria optimization problem that is adjustable via a plurality of parameters;
perform treatment planning optimization for delivery of the radiotherapy treatment, the treatment planning optimization comprising:
identifying parameterized linear programming equations from the radiotherapy problem;
converting the parameterized linear programming equations for execution by parallel processing hardware; and
solving a plurality of the converted parameterized linear programming equations in parallel on the parallel processing hardware, to produce a plurality of solutions to the radiotherapy problem corresponding to the plurality of parameters that define the radiotherapy problem; and
generate treatment plan data based on at least one of the plurality of solutions, wherein the treatment plan data is used to generate a radiation therapy treatment plan.

19. A computing system configured for radiotherapy treatment planning, the system comprising:
one or more memory devices to store data of a radiotherapy problem for providing radiotherapy treatment to a human subject from a radiotherapy treatment machine, the radiotherapy problem being a multicriteria optimization problem that is adjustable via a plurality of parameters; and
one or more processors configured to perform operations to:
perform treatment planning optimization for delivery of the radiotherapy treatment, the treatment planning optimization including operations to:
identify parameterized linear programming equations from the radiotherapy problem;
convert the parameterized linear programming equations for execution by parallel processing hardware; and
solve a plurality of the converted parameterized linear programming equations in parallel on the parallel processing hardware, to produce a plurality of solutions to the radiotherapy problem corresponding to the plurality of parameters that define the radiotherapy problem; and
generate treatment plan data based on at least one of the plurality of solutions,
wherein the treatment plan data is used to generate a radiation therapy treatment plan.

20-25. (canceled)

26. The computing system of claim 19, wherein the parallel processing hardware comprises a plurality of graphics processing units (GPUs).

27-35. (canceled)
